(19) United States
(12) Patent Application Publication
(10) Pub. No.: US 2022/0031390 A1
(43) Pub. Date: Feb. 3, 2022

(54) BIPOLAR TOOL FOR SEPARATING TISSUE ADHESIONS OR TUNNELING

(71) Applicant: Medtronic, Inc., Minneapolis, MN (US)

(72) Inventors: Garrett P. Ebersole, Hamden, CT (US); Saumya Banerjee, Hamden, CT (US); Jacob C. Baril, Norwalk, CT (US); Justin J. Thomas, New Haven, CT (US); Roy J. Pilletere, Middletown, CT (US); Matthew A. Dinino, Newington, CT (US)

(21) Appl. No.: 17/444,076

(22) Filed: Jul. 30, 2021

Related U.S. Application Data
(60) Provisional application No. 63/059,291, filed on Jul. 31, 2020.

(51) Int. Cl. A61B 18/14; A61B 1/00; A61B 1/05

(52) U.S. Cl. CPC A61B 18/1492; A61B 1/00087; A61B 2018/126; A61B 1/05; A61B 1/00096

(57) ABSTRACT

A bipolar tunneling tool is configured to separate tissue adhesions and tunnel. The bipolar tunneling tool includes a handle, a tunneling shaft, a return electrode, an optical window, an active electrode, and an insulative housing. The tunneling shaft extends from the handle. The return electrode is disposed proximate a distal end of the tunneling shaft. The optical window is disposed proximate the distal end of the tunneling shaft, the optical window defining a channel. The active electrode is at least partially disposed within the channel. The active electrode is configured to dissect a tissue and cauterize the tissue. The insulative housing is configured to electrically insulate the active electrode from the return electrode.

This application claims the benefit of U.S. Provisional Application Ser. No. 63/059,291, filed Jul. 31, 2020, the entire content of which is incorporated herein by reference.

### TECHNICAL FIELD

The present disclosure relates to bipolar electrosurgical instruments and, more particularly, to bipolar electrosurgical instruments for separating tissue adhesions or tunneling.

### BACKGROUND

Some medical procedures may include crossing multiple tissue layers to gain access to a location within the body of a patient. Such medical procedures may include implanting one or more medical devices or components thereof, e.g., medical electrical leads, at the location. One manner of accessing an intrathoracic location is substernally and includes traversing one or more layers of tissue, e.g., diaphragmatic attachments that attach the diaphragm to the sternum. An example of a procedure is the implantation of the distal portions of one or more leads substernally, and may include using an implant tool to access the intrathoracic cavity of the patient. The one or more leads may be part of an implantable cardiac system (e.g., implantable cardioverter-defibrillator (ICD)) that may be used to deliver electrical stimulation pulses to the patient's heart to terminate cardiac arrhythmias, such as ventricular fibrillation, bradycardia, or the like. Such implantable cardiac systems may include, or may be part of a system that includes, a subcutaneously-implantable housing that encloses a pulse generator or other electronics. The housing of some cardiac systems may be connected to the one or more leads, which may be configured to deliver defibrillation and/or pacing pulses.

In order to gain access to the heart for various heart-related procedures, a physician typically performs a sternotomy. A sternotomy is a surgical procedure that allows a doctor to reach the heart (or nearby organs) and blood vessels by first making an incision in the skin over the breastplate or sternum and then cutting through the sternum. A rib spreader may be utilized to expand the ribcage to gain internal access to the organs. When surgery is finished, the doctor repositions the sternum and uses a combination of staples and wire to secure the sternum in place while the patient heals. Over time, scar tissue and adhesions form to maintain the sternum in place as the patient returns to normal life. Surgeons may be hesitant to perform procedures requiring substernal access, such as those required to implant portions of an implantable cardiac stimulation system intrathoracically, on patients that have had a previous sternotomy.

### SUMMARY

This disclosure provides tools and implant techniques utilizing such tools to gain access to spaces within a patient, such as an extravascular space, e.g., to facilitate implantation of medical devices or components thereof, such as a lead, within the space. The tools may include one or more features that provide advantages, e.g., with respect to safety, during such procedures. These features may be particularly advantageous in the case of patients that have had a previous sternotomy but require intrathoracic implantation of medical devices or components thereof.

For example, the tools may include a curved shape to control the depth of the tool within the patient. As another example, the tool may include optical components to facilitate visualization during the procedure. As another example the tool may include a bipolar electrode pair that facilitates dissection and cauterization of tissue, while controlling the current field within the patient.

In some examples, a bipolar tunneling tool comprises: a handle; a tunneling shaft extending from the handle; a return electrode disposed proximate a distal end of the tunneling shaft; an optical window disposed proximate the distal end of the tunneling shaft, the optical window defining a channel; an active electrode at least partially disposed within the channel, the active electrode configured to dissect a tissue and cauterize the tissue; and an insulative housing configured to electrically insulate the active electrode from the return electrode.

In some examples, a bipolar tunneling tool comprises: a handle comprising an activation element; a tunneling shaft extending from the handle; a return electrode disposed proximate a distal end of the tunneling shaft; an active electrode disposed at the distal end of the tunneling shaft, wherein the activation element is configured to activate the active electrode in response to actuation of the activation element, and wherein the active electrode is configured to dissect a tissue and cauterize the tissue; an insulative housing configured to electrically insulate the active electrode from the return electrode; and a guide member extending from the handle in a substantially parallel fashion to the tunneling shaft, the guide member configured to move above an outer surface of a sternum in an extracorporeal fashion during use of the bipolar tunneling tool.

In some examples, a method comprises: inserting a tunneling shaft of a bipolar tunneling tool into a substernal space of a patient, wherein the bipolar tunneling tool comprises: a return electrode disposed proximate a distal end of the tunneling shaft; an optical window disposed proximate the return electrode, the optical window defining a channel; an active electrode at least partially disposed within the channel, the active electrode configured to dissect a tissue and cauterize the tissue; an insulative housing configured to electrically insulate the active electrode from the return electrode; and a camera disposed proximate the optical window and configured to connect, to a display; actuating an activation element configured to activate the active electrode in response to actuation of the activation element; and visualizing a dissection path of the bipolar tunneling tool using the camera.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

The following drawings are illustrative of example embodiments and do not limit the scope of the invention. The drawings are not to scale (unless so stated) and are intended for use in conjunction with the explanations in the following detailed description. Examples will hereinafter be described in conjunction with the appended drawings wherein like numerals denote like elements.

FIGS. 1A-B are conceptual diagrams illustrating an exemplary extravascular implant.

FIG. 2 is a conceptual diagram illustrating a sub-sternal access.

FIGS. 3-6 are conceptual diagrams illustrating an example bipolar tunneling device in accordance with the present disclosure.

FIG. 7 is a conceptual diagram of an example introducer sheath that may be used in conjunction with a bipolar tunneling device in accordance with the present disclosure.

FIGS. 8A-8D is a conceptual diagram illustrating an example bipolar tunneling device being advanced superiorly beneath a sternum of the patient in accordance with the present disclosure.

FIGS. 9A-C are conceptual diagrams illustrating example bipolar tunneling devices in accordance with the present disclosure.

FIG. 10 is a conceptual diagram illustrating a distal end of an example bipolar tunneling device in accordance with the present disclosure.

FIG. 11 is a conceptual diagram illustrating an example arrangement of a lighting element disposed proximate a camera for use with the bipolar tunneling device in accordance with the present disclosure.

FIG. 12 is a flow diagram of an example technique for operating the bipolar tunneling device.

The details of one or more examples of this disclosure are set forth in the accompanying drawings and the description below. Other features, objects, and advantages of this disclosure will be apparent from the description and drawings, and from the claims.

### DETAILED DESCRIPTION

The following detailed description is exemplary in nature and is not intended to limit, in any way, the scope, applicability, or configuration of the tools and techniques described in this disclosure. Rather, the following description provides practical examples, and those skilled in the art will recognize that some of the examples may have suitable alternatives.

This disclosure describes a bipolar tunneling tool that includes an electrosurgical implement configured to cauterize or dissect tissue. The handle of the bipolar tunneling tool may include a trigger that, when actuated, activates one or more electrodes disposed proximate a distal end of the tunneling shaft. In some examples, the electrodes include an active electrode and a return electrode. The active electrode may be configured to dissect tissue to promote advancement of the tunneling shaft along a desired dissection path; the active electrode may also be configured to cauterize tissue to stem bleeding. The return electrode may be configured to provide a return path during electrical activation of the active electrode to complete an electrical circuit. The active electrode may be secured by an insulative housing that electrically insulates the active electrode from the return electrode.

An optical window disposed at a distal end of the tunneling shaft may be configured to allow viewing therethrough to facilitate guidance of the bipolar tunneling tool along a desired dissection path. For example, a lighting element disposed within the tunneling shaft may emit light that passes through the optical window, illuminating an internal cavity of the patient. The emitted light may then return through the optical window to reach a camera (or other optical device) disposed within the tunneling shaft. In some cases, the lighting element is a fiber optic array, and the fiber optic array is configured to surround (e.g., encircle) the camera.

To maintain visualization via the camera of the bipolar tunneling tool, the active electrode may help prevent fluid from obstructing the optical window. For instance, if dissection of tissue results in bleeding, the active electrode may cauterize the tissue to stem bleeding. Accordingly, the active electrode coupled with the return electrode may facilitate dissection and coagulation by enabling the bipolar tunneling tool to more quickly and easily separate scar tissue, and the guide member, optical window, lighting element, and/or camera may allow the user to visualize the dissection during use of bipolar tunneling tool.

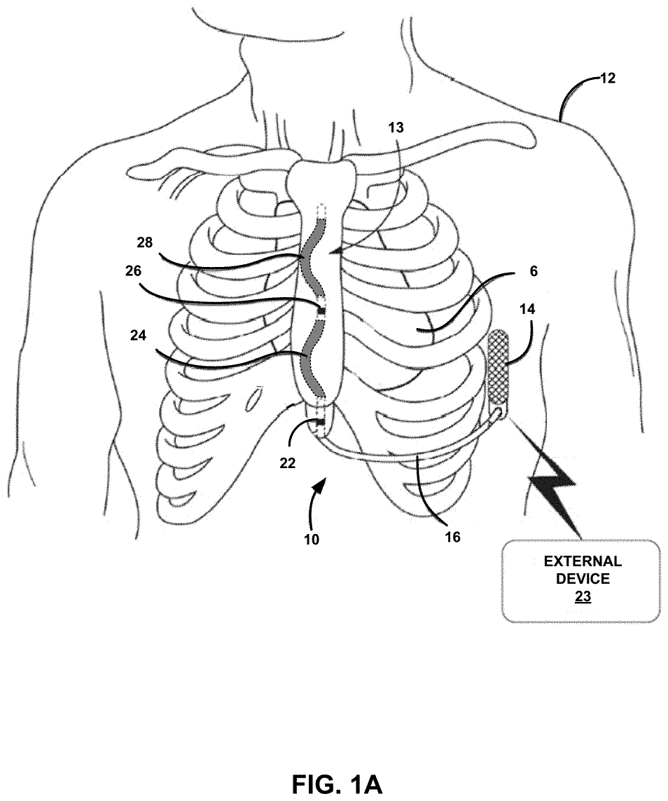

FIGS. 1A-B are schematics showing an exemplary extravascular implant of an exemplary system 10 that includes a pulse generator 14 and an implantable medical electrical lead 16 coupled thereto. Pulse generator 14 is shown implanted subcutaneously on the left mid-axillary of a patient 12, superficially of the patient's ribcage. Pulse generator 14, which may be configured to provide cardiac pacing and/or defibrillation therapy, includes a hermetically sealed housing in which the appropriate electronics and a power supply are contained, and which is formed from a conductive material, such as titanium, or from a combination of conductive and non-conductive materials. Pulse generator 14 may be in wireless communication with an external device 23 (e.g., a computing device for use by a patient, a clinician, etc.) to transmit information to external device 23, be programmed by external device 23, etc. Pulse generator 14 further includes a connector module by which lead 16 is electrically coupled to the electronics contained therein, for example, by electrical contacts contained within the connector module and a corresponding hermetically sealed feedthrough assembly, such as is known in the art. The conductive material of device housing may be employed as an electrode, for example, to provide the aforementioned therapy in conjunction with one or more pace/sense electrodes 22, 26 and/or defibrillation electrodes 24, 28 of lead 16, which is shown implanted in a sub-sternal space 3, for example, within the loose connective tissue and/or sub-sternal musculature of the anterior mediastinum.

Lead 16 may have any of a number of configurations. For example, lead 16 may include more or fewer pace/sense electrodes. In another example, lead 16 may include more or less than two defibrillation electrodes 24, 28 and/or have a defibrillation electrode(s) that is formed of multiple segments. Examples of leads with multiple defibrillation electrodes and/or segments are described in commonly assigned, co-pending U.S. Patent Publication No. 2015/0306375 (Marshall et al.), U.S. Patent Publication No. 2015/0306410 (Marshall et al.) and U.S. Patent Publication No. 2016/0158567 (Marshall et al.), each of which is incorporated herein by reference in its entirety.

With reference to FIG. 1B, sub-sternal space 3 may be viewed as being bounded laterally by pleurae 39 that enclose the patient's lungs, posteriorly by the pericardial sac 15 that encloses the patient's heart 6, and anteriorly by a sternum 13. In some instances, the anterior wall of the anterior mediastinum may also be formed by the transversus thoracis and one or more costal cartilages. Although FIG. 1A and 1B are described in the context of the distal portion of lead 16 being placed within sub-sternal space 3, in other examples, the tools and implant techniques described herein may be used to implant a distal portion of lead 16 at other locations outside the heart. In one example, the tools may be used to place the distal portion of lead 16 intra-pericardially via a percutaneous subxiphoid approach. In some examples, the tools and implant techniques described herein could be used for implanting other medical devices or components thereof and/or for other spaces within the patient, such as, implanting a leadless pacemaker on or near outside of heart via substernal access.

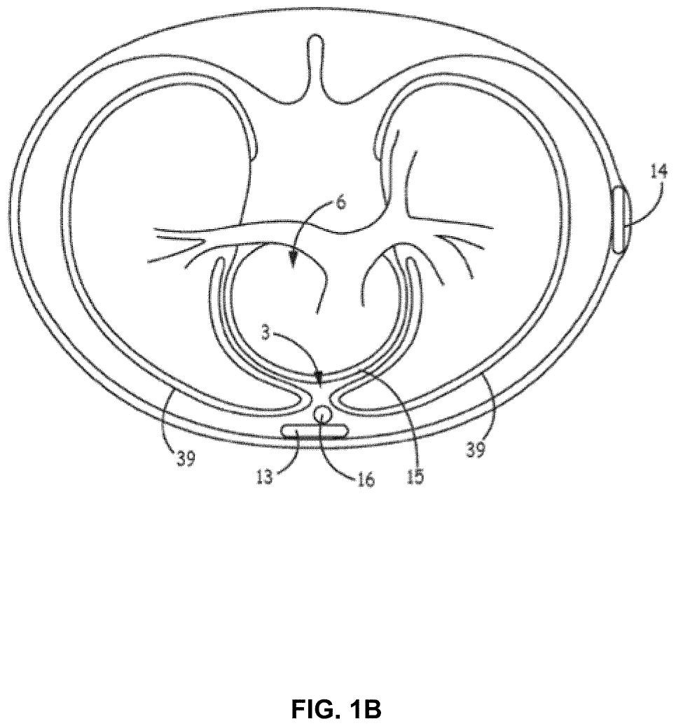

FIG. 2 is a schematic showing an access site A for making a passageway between a patient's diaphragm 19 and xiphoid process 20 of sternum 13, for example, to create a sub-sternal tunnel in which to position a medical device, such as medical electrical lead 16. After making a superficial incision, an operator, using a tunneling tool, may open a passageway between diaphragmatic attachments 18 and diaphragm 19, for example, by blunt and/or sharp dissection, in which the operator may employ a tunneling tool, such as those example tools described herein, to both create the passageway and then form a sub-sternal tunnel (e.g. along the dotted line of FIG. 2). Because the bony structure of sternum 13 inhibits external palpation, the operator may need to take extra care, during the dissection (e.g., blunt and/or sharp) and/or tunneling, not to injure sub-sternal structures or the chest cavity, which could compromise the pleura of the lungs or heart 6. Tools and associated methods disclosed herein are configured to help an operator gain the desired sub-sternal access and create a space in which to position a medical device, such as medical electrical lead 16, in a controlled fashion that mitigates the risk of injuring bodily organs.

In some examples, a distal portion of lead 16 may be implanted to be located between the posterior aspect of sternum 13 and the anterior wall of heart 6. The implant procedure may be performed by using a blunt trocar with a flexible port to create a small tunnel near the posterior aspect of sternum 13 via entry into the body near xiphoid process 20. The distal portion of lead 16, e.g., the portion of lead 16 carrying some or all of electrodes 22, 24, 26, 28, is then placed in the anterior mediastinum. The proximal end of lead 16 is then tunneled subcutaneously or submuscularly to a left midxillary location under and connected to the pulse generator 14. Pulse generator 14 and lead 16 are able to provide, e.g., defibrillation, anti-tachycardia pacing (ATP), bradycardia pacing, post-shock pacing, and asystole “lifeboat pacing.”

Suitable tunneling tools with blunt trocars may be utilized to implant lead 16 in patient 12. However, patients that have had previous median sternotomies tend to have extensive scar tissue and the pericardium is often adhered to the posterior of the sternum. This scar tissue makes it very challenging and, in some cases, relatively undesirable for a blunt trocar to create a small tunnel in the anterior mediastinum. A surgeon may not want to enter the substernal space of such patients unless they have direct visualization of the location. Thus, patients that have had one or more previous median sternotomies may be less likely to receive an extravascular ICD or other medical device that includes the implantation of distal portion of lead 16 between the posterior aspect of sternum 13 and the anterior wall of the heart 6 in the manner described above.

In accordance with some examples of the disclosure, a tunneling tool (or trocar) is described that allows for a forward, hemispherical view and selective dissection (e.g., selective between blunt and sharp dissection) of tissue while tunneling through diaphragmatic attachments, pericardial adhesion, and other soft tissue. Examples include tunneling tools having a knife blade or other cutting tool with a sharp edge, e.g., on the distal end of the tool, for accessing the substernal space, dissection adhesions, and creating working room, e.g., in the thoracic cavity. Such tunneling tools may also include an optical window for a surgeon or other user to visualize, e.g., using an endoscope inserted within the tunneling tool, the movement of a distal end of the tunneling tool through tissue. Such visualization may provide for better guidance of the tunneling tool during an implant procedure and allow for a clinician to identify locations in which it may be desirable to deploy the cutting tool, e.g., to cut tissue adjacent the distal end of the tunneling tool using sharp dissection rather than blunt dissection. Such tunneling tools may also include a bipolar electrode pair that facilitates cauterization during dissection of the tissue. In some examples, the cutting tool comprises or acts as one of the electrodes of the bipolar pair.

Examples of the disclosure may provide tools that allow for blunt dissection/tunneling as well as transection of adhesions under direct visualization. Such a tool enables safe placing of extravascular ICDs even in patients with previous sternotomies. The optical window with integrated knife or other cutting blade may allow for easy identification of tissue prior to dissection during a tunneling procedure. Examples of the disclosure may also allow for easier access into the thorax of a patient with previous sternotomies for a coronary artery bypass graft (CABG) or valve replacement. The example tool may allow for the reduction of pericardial adhesions to create working space for placement, adjustment, and removal of lead,

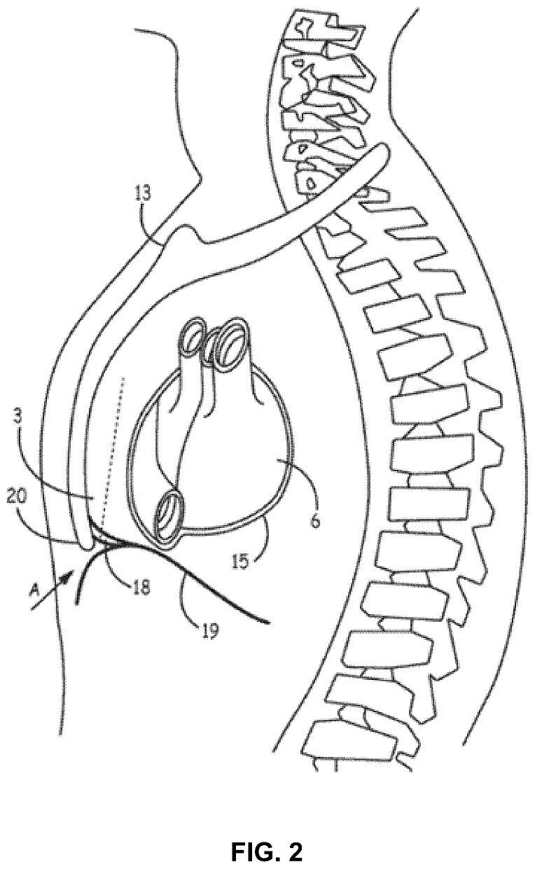

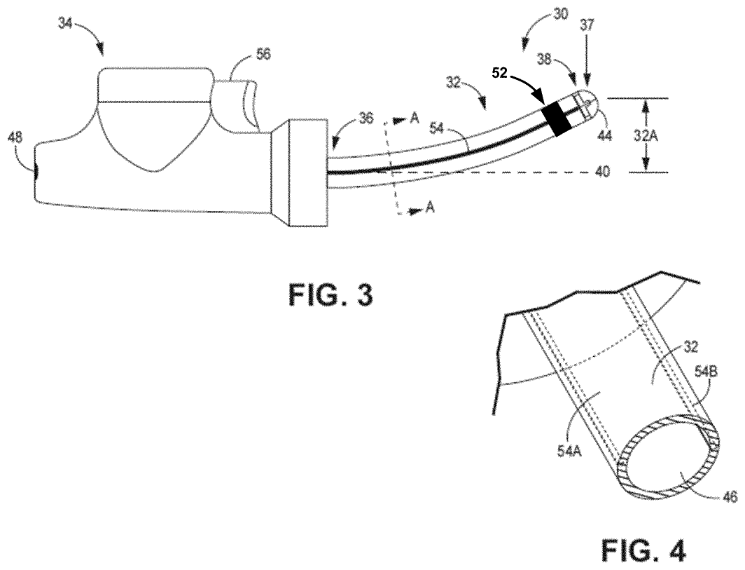

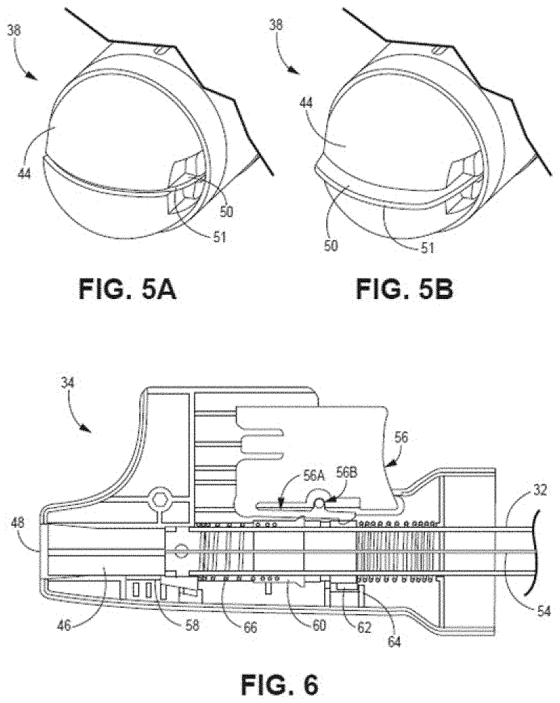

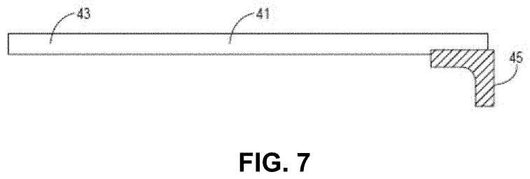

FIGS. 3-6 are functional schematic diagrams illustrating an example bipolar tunneling tool 30 for gaining sub-sternal access and creating a sub-sternal tunnel in a patient, according to some examples. FIG. 3 illustrates bipolar tunneling tool 30 including a tunneling shaft 32 and a handle 34. FIG. 4 illustrates a cross-section view of tunneling shaft 32 about cross-section A-A shown in FIG. 3. FIGS. 5A and 5B illustrate a distal end 38 of tunneling shaft 32. FIG. 6 illustrates a cross-sectional view of handle 34.

As shown in FIG. 3, tunneling shaft 32 of bipolar tunneling tool 30 extends from proximal end 36 to distal end 38 (or “distal tip 38”). Bipolar tunneling tool 30 also includes handle 34, which is shown coupled to proximal end 36 of tunneling shaft 32. Tunneling shaft 32 may extend from proximal end 36 to distal end 38 in a linear or straight manner. In some examples, as shown in FIG. 3, at least a portion of tunneling shaft 32 extends in a curved orientation from proximal end 36 to distal end 38, e.g., relative to an axis 40. Axis 40 may be defined by a central longitudinal axis of handle 34 or may be defined by a portion of shaft 32 that extends initially from handle 34 in a substantially straight manner before exhibiting a curved orientation beginning at a point between proximal end 36 and distal end 38 of shaft 32. The curved orientation of tunneling shaft 32 results in offset 32A between distal end 38 and axis 40 shown in FIG. 3. Offset 32A may range from approximately 0.35 inches to approximately 1.25 inches, such as, approximately 0.720 inches, although other examples are contemplated. In some instances, the curvature of tunneling shaft 32 may maintain the path of the distal tip close to posterior side of sternum and away from vital organs like lung or heart during a tunneling procedure.

In some examples, tunneling shaft 32 be curved about the entire length from proximal end 36 to distal end 38 (e.g., as shown in FIG. 3) or may include one or more sections that are substantially straight with one or more other sections that are curved. For example, a proximately portion of the tunneling shaft 32 extending directly from handle 34 may be approximately straight for some of the length of tunneling shaft 32 and then transition to a more distal portion of tunneling shaft 32 that is curved. In some examples, the curved portion of tunneling shaft exhibits a radius of curvature of about 15 inches to about 40 inches.

Tunneling shaft 32 may be tubular, e.g., have a circular or oval outer profile and/or define one or more inner lumens, as shown in FIGS. 4-6. Any suitable material may be used for tunneling shaft 32, e.g., metals (stainless steel, coated steel, titanium alloys, aluminum alloys and others) and plastics (unfilled and filled with suitable fiber like glass or carbon for strength and rigidity) may be utilized. Suitable plastic materials include but are not limited to acetal copolymer, polytetrafluoroethylene (PTFE) (e.g., TEFLON), polyether ether ketone (PEEK), polyphenylsulfone (PPS15) (e.g., RADEL), and polycarbonate. In some examples, tunneling shaft 32 may be formed of a material that allows for all or at least a portion of tunneling shaft 32 to be transparent along the length of shaft 32. Shaft 32 may be substantially rigid so the clinician can control accurately the position of the tip in relation to vital organs under visualization afforded by fluoroscopy or other techniques via an optical window 44. To that end, in some examples, the tip of tunneling shaft 32 preferably has at least some metal components (like a metal blade) which will allow visualization using suitable medical imaging technology.

In some examples, rigidity of tunneling shaft 32 may be described in the context of possible forces that may act of tunneling shaft 32, e.g., during an implant procedure. As one example, an operator may apply a force to keep tunneling shaft 32 (e.g., distal end 38) pressed against sternum 13 of patient 12. The rigidity of tunneling shaft 32 may be such that tunneling shaft 32 does not flex significantly when the operator is biasing tunneling shaft 32 upwards/anteriorly. In some examples, tunneling shaft 32 exhibits substantially no flex when greater than about 5 pounds of force is applied to distal end 38 in direction 37 shown in FIG. 3. In some examples, tunneling shaft 32 does not “jam up” (e.g. can still deploy an active electrode 50 and/or allow for visualization via an optical window 44) when greater than about 5 pounds of force is applied to distal end 38 in direction 37 shown in FIG. 3.

Another example force that may act on shaft 32 is a torque on shaft 32 when an operator is trying to keep shaft 32 aligned during insertion. If the operator is rotating shaft 32 back and forth along the axis 40, shaft 32 must have substantial rigidity to sweep back and forth on the posterior side of sternum 13, clear away adhesions, and still “fire” without jamming (e.g., still deploy active electrode 50) on the order of about 5 inches*pound of torque.

Tunneling shaft 32 may exhibit any suitable shape and dimensions. While FIG. 4 shows that tunneling shaft 32 has a substantially circular cross-section, other example cross-section shapes are contemplated. For example, as described further below, in some examples, tunneling shaft 32 may exhibit an oval cross-section. The outer diameter (in the case of a circular cross-section) or greatest outer dimension (in the case of a non-circular cross-section) of tunneling shaft 32 may range from about 3 millimeters (mm) to about 15 mm, although other examples are contemplated. The length of tunneling shaft 32 from proximal end 36 directly adjacent handle 34 to distal end 38 may range from about 4 inches to about 12 inches, although other examples are contemplated. In examples in which a portion of tunneling shaft 32 is substantially straight from the proximal end 36 adjacent to handle and then transitions to a curved portion at a point between proximal end 36 and distal end 38, approximately ⅓ (one-third) of the length of tunneling shaft 32 out of proximal end 36 may be approximately straight. In some examples, tunneling shaft 32 may have a portion that is approximately straight for a length of about 0.5 inches to about 1.5 inches (e.g., in the case of tunneling shaft 32 having an overall length of about 4 inches). In some examples, tunneling shaft 32 may have a portion that is approximately straight for a length of about 3 inches to about 5 inches (e.g., in the case of tunneling shaft 32 having an overall length of about 12 inches). In some examples, approximately ⅔ (two-thirds) of the overall length of tunneling shaft 32 out of proximal end 36 may be approximately straight. In some examples, tunneling shaft 32 may have a portion that is approximately straight for a length of about 7 inches to about 9 inches (e.g., in the case of tunneling shaft 32 having an overall length of about 12 inches).

Tunneling shaft 32 defines an inner lumen 46 that extends from the proximal end 36 to distal end 38. As shown in FIG. 6, inner lumen 46 runs from tunneling shaft 32 through handle 34, terminating at proximal opening 48 for handle 34. Distal end 38 of tunneling tool 32, also include optical window 44 that is shaped to allow for blunt dissection when tunneled through tissue of patient 12, e.g., using one or more of the techniques described herein. In the example of FIGS. 3-6, optical window 44 has a dome shape for the leading edge to allow for blunt dissection. However, other shapes are contemplated. Optical window 44 may he formed of a transparent material, for example glass, quartz or clear plastics like polycarbonate (e.g., LEXAN) or acrylic. During an implant procedure, an endoscope or other optical tool may be inserted into inner lumen 46 via proximal opening 48 in handle 34 and advanced through lumen 46 to distal end 38 of tunneling tool 32 adjacent optical window 44. In this manner, a surgeon or other user may visualize the path of distal end 38 when advanced through tissue of patient 12 during the insertion of bipolar tunnel tool 32 into patient 12.

Additionally, as shown in FIGS. 5A and 5B, bipolar tunneling tool 30 includes active electrode 50 at distal end 38. Active electrode 50 may take the form of a knife blade, scalpel blade, or other tool with a sharp edge 51 that is configured to cut through tissue, such as, scar tissue, of patient 12 while bipolar tunneling tool 30 is tunneled in the sub-sternal space 3 of patient 12 to a target location. Active electrode 50 may be configured to be selectively actuated by a surgeon or other user from a recessed position (as shown in FIG. 5A) to a deployed position (as shown in FIG. 5B). Distal end 38 may be generally blunt when active electrode 50 is in a recessed position, e.g., to prevent wounding of vital organs like mammary arteries or lungs during a tunneling procedures. Distal end 38 may include a beveled tip with optical window 44. However, other distal end geometries are contemplated. For example, distal end 38 may include a domed or sphere-shaped tip, which may be made of a clear or otherwise transparent material.

When active electrode 50 is in the recessed position, the lead or cutting edge of active electrode 50 is recessed into distal end 38 of tunneling shaft 32 such that the outer surface of optical window 44 defines the leading edge of the tool, allowing for blunt dissection of tissue while tunneling shaft 32 is advanced in sub-sternal space of tissue. Conversely, when active electrode 50 is in the deployed position, active electrode 50 defines the leading edge of the tunneling shaft 32, allowing for tissue, such as, scar tissue, to be cut by the tool by sharp dissection rather than be bluntly dissected. Active electrode 50 may have any shape suitable for dissection, e.g., a semi-circular blade. In some examples, active electrode 50 is welded or otherwise attached in a perpendicular arrangement relative to distal end 38.

Bipolar tunneling tool 30 includes a return electrode 52 disposed proximate distal end 38. Return electrode 52 is configured to provide a return path during electrical activation of active electrode 50 to complete an electrical circuit. For example, distal end 38 may define return electrode 52 in the shape of a band or a cuff, in this way allowing for a robust 360° return path. In other examples return electrode 52 may have different sizes or shapes. Return electrode 52 may be adapted to connect to pulse generator 14. An activation element, such as a trigger 56 of handle 34 may be selectively actuated by a user to initiate bipolar electrosurgical energy to active electrode 50 and return electrode 52.

Handle 34 may be shaped such that when gripped by a hand of a surgeon or other operator, trigger 56 may be depressed or otherwise actuated a finger such as the index finger in the case of bipolar tunneling tool 30. In other examples, handle 34 may be shaped such that when gripped by a hand of a surgeon or other operator, trigger 56 may be depressed or otherwise actuated by the thumb. In some examples, trigger 56 may be shielded by adjacent walls of handle 34 by recessing trigger 56 to some extent into the surface of handle, e.g., to protect against unwanted depression of trigger 56 during a tunneling procedure. In some examples, handle 34 may include a trigger guard to prevent trigger 56 from being depressed accidently by a surgeon or other operator during a tunneling procedure. In some examples, a vertical portion of handle 34 may be angled further towards distal end 38 of tunneling shaft 32.

Any suitable mechanism may be utilized to allow for active electrode 50 to be transitioned between the recessed position (FIG. 5A) and deployed position (FIG. 5B). For example, as shown in FIGS. 3, 4, and 6, tunneling shaft 32 includes blade arms 54A and 54B (collectively “blade arms 54”) parallel to the plane of leading edge 51 of active electrode 50 within tunneling shaft 32, which are coupled to active electrode 50 at the distal end and extend back to handle 34. Blade arms 54 may be located within tunneling shaft 32, inside inner lumen 46 and adjacent to the inner wall of tunneling shaft 32, and/or adjacent to the outer surface of tunneling shaft 32 (e.g., within tracks recessed into tunneling shaft 32). In the example in which blade arms 54 are located adjacent the outer surface of tunneling shaft 32 (e.g., within recessed tracks), bipolar tunneling tool 30 may include an outer sheath, e.g., a thin shrink wrap, that assists in securing blade arms in place relative to tunneling shaft 32.

Blade arms 54 are mechanically coupled to handle 34 such that the actuation of trigger 56 translates blade arms 54 along curved shaft 32 towards distal end 38 of bipolar tunneling tool 30 to transfer mechanical energy to active electrode 50 to actuate active electrode 50 from the recessed position to the deployed position. In the deployed position, sharp/leading edge 51 of active electrode 50 may extend about 0.25 mm to about 2 mm beyond distal end 38 of tunneling shaft 32. Put another way, in the deployed position, sharp/leading edge 51 of active electrode 50 may extend about 0.25 mm to about 2 mm beyond the leading edge of distal end 38, e.g., the outer surface of optical window 44, when active electrode 50 is in the recessed position.

In some examples, the depression (pulling) of trigger 56 actuates active electrode 50 from the recessed position to the deployed position and active electrode 50 may remain in the deployed position until trigger 56 is released. Alternatively, tunneling member 30 may be configured such that the actuation of trigger 56, e.g., depression or depression and release of trigger 56, may result in active electrode 50 being actuated from the recessed position to the deployed position and then automatically returned to the recessed position, e.g., after active electrode 50 advances forward a pre-set distance. In some examples, a surgeon or other user may hold handle 34 of bipolar tunneling tool 30 stationary when trigger 56 is depressed to control the length of tissue that is dissected by active electrode 50, which approximately corresponds to the length at which sharp/leading edge 51 of active electrode 50 extends out of distal end 38 when trigger 56 is depressed to move active electrode 50 into the deployed position. Alternatively, or additionally, a surgeon or other user may advance tunneling shaft 32 forward by handle 34 when active electrode 50 is held in the deployed position, where the length of tissue dissection by active electrode 50 corresponds generally to the length that tunneling shaft 32 is advanced under the control of the surgeon or other user.

As one example, in the configuration shown in FIG. 6, drive arms 54 are connected to a spring/hammer/bushing mechanism in handle 34 that includes scope retention slot 58, hammer 60, blade bushing 62 and bushing stop 64. When trigger 56 is pulled, hammer 60 is retracted against spring 66. The trigger 56 has a cantilever beam 56A with a hook. The hook engages the hammer 60 and drives it against spring 66. The beam 56A has also the post 56B interacting with the slot in the wall of the handle body (not shown). When the trigger 56B is advanced sufficiently, the pin 56B, guided by the slot raises the hook and releases the hammer 60. Upon release, hammer 6( )springs forward impacting blade bushing 60 to transmit mechanical energy to blade drive arms 54 until impacting a stop point defined by bushing hard stop 64. Bushing hard stop 64 in handle 34 prevents blade bushing 62 from advancing active electrode 50 beyond a safe distance out of distal end 38 of tunneling shaft 32.

Additional configurations of tunneling tools and mechanisms for transitioning a cutting tool from the recessed position to the deployed position are described in U.S. Patent Application Publication No. 2020/0038048 (Ebersole et al.), which is incorporated herein by reference in its entirety.

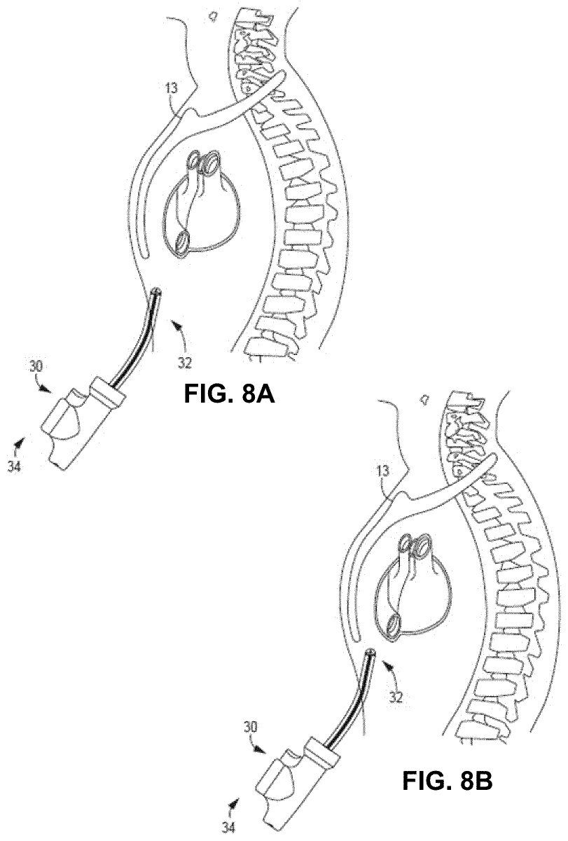

During a procedure to gain sub-sternal access and create a sub-sternal tunnel in a patient, e.g., to implant a medical device such as lead 16, tunneling shall 32 may be inserted into the inner lumen of an introducer sheath, e.g., wherein the sheath is sized to extend from approximately distal end 38 to approximately proximal end 36 of tunneling shaft 32 prior to insertion and advancement of tunneling shaft 32 in patient 12. An example of an introducer sheath 41 is illustrated in FIG. 7. Sheath 41 includes a body 43 and a handle 45. Body 43 of sheath 41 defines an inner channel. In one example, sheath 41 may be an open sheath as illustrated and described in U.S. patent application Ser. No. 14/196,298 and U.S. patent application Ser. No. 14/196,443, both of which are incorporated herein by reference in their entireties. In the case of an open sheath, sheath 41 may include an opening along the length of body 43 and the inner channel is accessible via the opening anywhere along the length of body 43. In another example, sheath 41 may be a splittable sheath in which body 43 includes a score or other weakened portion to permit splitting of body 43, e.g., as illustrated and described in further detail in U.S. patent application Ser. No. 14/196,443, previously incorporated above. In yet another example, sheath 41 may be a sheath without any gap or score on body 43, in which case sheath 41 may be removed by slitting the sheath using a slitter, as illustrated and described in U.S. patent application Ser. No. 14/196,443, previously incorporated above. Sheath 41 may have other properties describe above in reference to U.S. patent application Ser. No. 14/196,298 and U.S. patent application Ser. No. 14/196,443 or any commercially available sheaths.

The distal portion of the introducer sheath 41 may have an open end so as to no obstruct the view through optical window 44 and deployment of active electrode 50 at distal end 38 of tunneling shaft 32. Also, the open end allows for insertion of a lead to the targeted area. Once tunneling shaft 32 is inserted into the introducer sheath 41, distal end 38 may be inserted into an incision site, e.g., at access site A, and then tunneled superiorly to both create the passageway and then form a sub-sternal tunnel (e.g., along the dotted line of FIG. 2). During the tunneling, a surgeon or other operator may control the advancement and direction of tunneling shaft 32 by gripping handle 34, which is located external to the body of patient. The surgeon or other operator may view the path of distal end 38 of tunneling shaft 32 during the procedure through optical window 44 using an endoscope or other viewing device inserted within inner lumen 46 of shaft 32. The surgeon or other operator may tunnel through tissue of patient 12 by way of blunt dissection using distal end 38 of tunneling shaft with cutting tool in the recessed position. The surgeon or other operator may also selectively deploy active electrode 50, e.g., to cut scar tissue or other areas where blunt dissection (e.g., via the distal end 38 when active electrode 50 is recessed) does not allow for tunneling of tool shaft 32. Then, for example, tunneling shaft 32 of bipolar tunneling tool 30 is withdrawn from the patient's body, leaving the introducer sheath 41 within the sub-sternal tunnel. The operator may pass a medical device, such as the above described lead 16, through the sheath lumen, via a proximal opening of the introducer sheath. The surgeon or other operator may then remove the introducer sheath 41 from the body, leaving lead 16 within the sub-sternal tunnel, and then remove the sheath from the lead for example, by slitting or splitting the introducer sheath from around lead, according to some embodiments and methods.

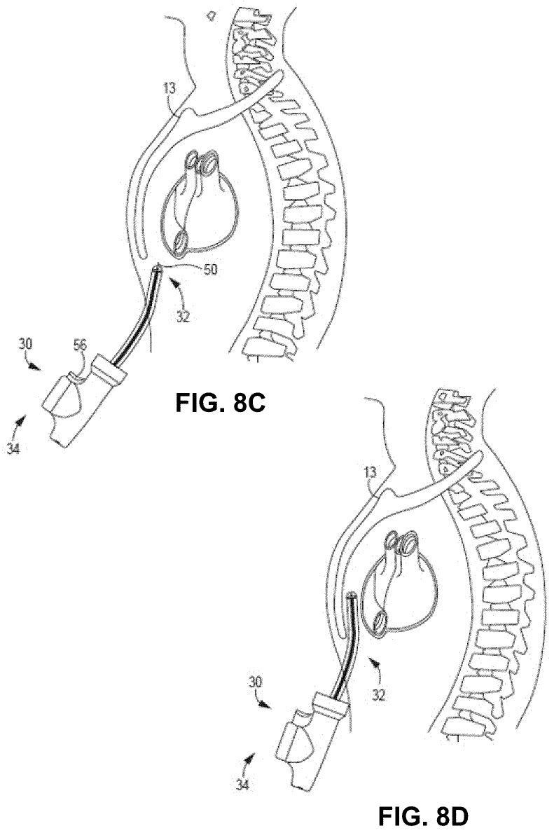

FIGS. 8A-8D are conceptual diagrams illustrating a progression of bipolar tunneling tool 30 during an example tunneling technique in accordance with the disclosure to insert at least a portion of shaft 32 into the substernal space under sternum 13. As described herein, the surgeon or other operator may selectively deploy or recess active electrode 50 at distal end 38 of tunneling shaft 32 as desired during the tunneling procedure as well as visualize the tissue space through optical window 44 during the procedure.

As shown in FIG. 8A, a distal portion of tunneling shaft is inserted through an incision site, e.g., at access site A shown in FIG. 2, with active electrode 50 in the recessed position at distal end 38 of tunneling shaft 32, with the operator controlling the movement of shaft 32 by gripping handle 34, which is located externally. Handle 34 remains outside patient 12 to allow for a surgeon or other operator to maneuver tunneling shaft 32 along the desired path within the substernal space of patient 12. Distal end 38 of shaft 32 may be advanced superiorly, e.g., to the position shown in FIG. 8B, to create a portion of a passageway and a sub-sternal tunnel. The surgeon or other operator may view the path of distal end 38 of tunneling shaft 32 during the procedure through optical window 44 using an endoscope or other viewing device inserted within inner lumen 46 of shaft 32. The surgeon or other operator may tunnel through tissue of patient 12 by way of blunt dissection using distal end 38 of tunneling shaft with active electrode 50 in the recessed position.

At the position shown in FIG. 8B, an operator may determine that a tissue (e.g., a diaphragmatic attachment, pericardium, scar tissue, or connective tissue) is directly adjacent to distal end 38 of shaft 32, e.g., using optical window 44. At that position, the operator may deploy active electrode 50 to the deployed position (e.g., as shown in FIG. 8C) and activate active electrode 50 to cut and cauterize the tissue adjacent to distal end 38 of shall 32, e.g., via sharp dissection with the leading edge 51 of active electrode 50. The operator may deploy and activate active electrode 50 by depressing trigger 56. In some example, during the depression of trigger 56, the operator may hold handle 34 and, thus, shaft 32 generally in place while the cutting tool 51 is advanced out to distal end 38 to the deployed position to cut the adjacent tissue. Once the tissue has been cut by the deployment of active electrode 50, active electrode 50 may be retracted back to the recessed position (e.g., automatically or with the release to trigger 56) and then advanced by the operator past the cut tissue to the position shown in FIG. 8D, e.g., to provide a path for the placement of lead 16 in the anterior mediastinum.

As illustrated by FIGS. 8A-8D, an operator may tunnel or otherwise advance a distal portion of tunneling shaft 32 from the incision site to a desired location such that active electrode 50 is selectively deployed and activated, e.g., as needed cut through tissue such as, e.g., diaphragmatic attachment, pericardium, scar tissue, or connective tissue, adjacent to distal end 38 during the tunneling procedure. Depending on patient anatomy, there may be tissue in the anterior mediastinum or other anatomical location along the pathway of tunneling shaft 32. During initial insertion, tunneling shaft 32 may penetrate through diaphragmatic attachments that were not dissected with the initial incision. If a patient has not had a previous sternotomy or other procedure, the mediastinal tissue (pericardium, lungs) may move be moving freely. Open space may exist after creating the incision and air is introduced, but expansion of the lungs during breathing may fill this space. Bipolar tunneling tool 30 may be particularly useful for patients who have had a previous sternotomy or other open chest procedure. These patients may have severe adhesions to the posterior sternum as result. In such cases, the tissue may be a mixture of pericardium, scar tissue, or connective tissue that forms in response to the injury from the first surgery. When the distal portion of tunneling shaft 32 is introduced into these patients, tunneling shaft 32 may be tunneling and cutting (e.g., via selective deployment of active electrode 50) through this mixture of tissue in order to safely place a lead, such as lead 16, as desired within patient 12 using the example techniques described herein.

As shown in FIGS. 8A-8D, the curvature of tunneling shaft 32 is such that the distal portion of tunneling shaft 32 is biased towards in the inner surface of sternum 13. Additionally, the leading edge 51 of active electrode 50 (illustrated in FIG. 5B, e.g.) extends along a plane that is nonorthogonal (e.g., generally parallel) to the plane of sternum 13 and outer surface of pericardial sac adjacent tunneling shaft 32. Such a configuration may allow for blade arms 54 to be driven from a mechanism within handle 34 more easily, e.g., compared to a configuration in which leading edge 51 of active electrode 50 is rotated 90 degrees from that shown in FIG. 3.

Active electrode 50 may have any suitable orientation when employed by bipolar tunneling tool 30. In the example of FIGS. 3-6, the plane of leading/sharp edge 51 of active electrode 50 is oriented approximately parallel to the central longitudinal axis of handle 34 and direction in which trigger 56 is depressed to deploy active electrode 50. Additionally, active electrode 50 extends along a plane such that, during normal use tunneling through tissue in sub-sternal space 3 as described herein, active electrode 50 is oriented non-orthogonal (e.g., approximately parallel) to the sternum inner surface, pericardium, and/or heart of patient 12.

Active electrode 50 may comprise or be a part of an electrosurgery implement. In such an example, drive arms 54 may be configured to conduct current and insulated from tissue contact within tunneling shaft 32 when in a recessed position. An electrical connector may be located, e.g., be included in handle 34. In some examples, depressing trigger 56 may advance active electrode 50 out of distal end 38 temporarily to cauterize or dissect tissue. For example, a single actuation (e.g., a depression) of trigger 56 may cause active electrode 50 to advance out of distal end 38 and activate active electrode 50. In another example, a first actuation (e.g., a partial depression) of trigger 56 may cause active electrode 50 to advance out of distal end 38, and a second actuation (e.g., a complete depression) of trigger 56 may activate active electrode 50. In yet another example, actuation of trigger 56 may cause active electrode 50 to advance out of distal end 38, and actuation of a second element (e.g., a button, positioned on handle 34, configured to be actuated by a thumb or otherwise) may activate active electrode 50.

Activating active electrode 50 and return electrode 52 may break stubborn tissue areas and scar tissue. Activating active electrode 50 and return electrode 52 may also cauterize tissue, thereby stemming bleeding that would otherwise obstruct visualization via viewing window 44. The tissue is dissected proximate active electrode 50 and current is returned through return electrode 52, completing the electrical circuit. Trigger 56 (or some other activation element, such as a button, a switch, etc.) of handle 34 may be selectively actuated by a user to initiate bipolar electrosurgical energy to active electrode 50 and return electrode 52.

In some examples, bipolar tunneling tool 30 may include a single blade arm 54 that translates to transmit mechanical energy from handle 34 to active electrode 50 rather than two blade arms 54A, 54B as shown for bipolar tunneling tool 30. Single blade arm 54 may extend from handle 34 along curved tunneling shaft to active electrode 50 located at distal end 38 of tunneling tool 70. A single drive arm 54 may transmit mechanical energy from handle 34 to an adjacent side of active electrode 50 while the other side of active electrode 50 is anchored (e.g., via a pin) to a portion of tunneling shaft 32, such as, optical window 44. The transmitted energy causes active electrode 50 to rotate about the anchoring point to expose the sharp leading edge of active electrode 50 to tissue adjacent optical window 44.

In some examples, tunneling shaft 32 may include a first curved tube and second curved tube of generally matching curvature. The first tube may have a greater diameter than that of the diameter of the second tube such that second tube is nested, e.g., coaxially, within the first tube. The first tube and second tube may be sized and configured to be moveable relative each other. The movement of the first tube relative to the second tube may be actuated by depression of trigger 56 to selectively transition bipolar tunneling tool 30 between a recessed and deployed configuration. Actuation of trigger 56 may move the first tube distally while the second tube remains stationary. In other examples, the second tube is moveable with the actuation of trigger 56 while the first tube is stationary. In some example, tunneling tool 80 may include a lubricious layer located between the outer surface of the second tube and inner surface of the first tube to promote movement of tubes 82 and 84 relative to each other

In some examples, both the first tube and the second tube may be flexible. In some examples, bipolar tunneling tool 30 includes optical window 44 through which an endoscope or other optical device may employed to allow a surgeon or other user to visualize the space outside of distal end 38 of bipolar tunneling tool 30 during an implant procedure. Likewise, as described above, bipolar tunneling tool 80 may include a semi-circular active electrode 50 (e.g.,) that may be selectively actuated between a recessed and deployed position, e.g., to allow for tunneling tool 80 to be used for blunt and sharp dissection of tissue, respectively, during implantation in the sub-sternal space 3 as desired based on the tissue viewable via optical window 44.

In some examples, tunneling shaft 32 may have a substantially oval (e.g., elongated oval) cross-section in a plane orthogonal to the longitudinal axis of bipolar tunneling tool 30. The oval cross-sectional shape may allow for substantially flat surfaces (“top” and “bottom” surfaces) of tunneling shaft 32 to be adjacent to the sternum inner surface and pericardial sac outer surface when utilized according to the tunneling techniques described herein. In some example, the “flat upper” surface of shaft 32 may be guided or otherwise in contact with sternum inner surface during a tunneling procedure, which may provide for increase stability of bipolar tunneling tool 30 in the hands) of a surgeon or other operator. Tunneling shaft 32 includes inner lumen 46, blade arms 54, and one or more additional lumens extending from proximal end 36 to distal end 38 of tunneling shaft 32.

In examples where tunneling shaft 32 has a substantially oval cross-sectional shape, the leading edge of active electrode 50 may extend in a direction substantially perpendicular to the long axis of the oval cross-section of tunneling shaft 32, and may be located between an additional (or “working”) lumen. In some examples, leading edge 51 of active electrode 50 may extend in an angled, non-perpendicular direction relative to the long axis of the oval cross-section of tunneling shaft 32. The angled orientation of active electrode 50 may allow for the length of the active electrode 50 to be longer than, e.g., the length of active electrode 50 oriented perpendicular to the long axis of the oval cross-section, resulting in a larger cutting profile. Bipolar tunneling tool 30 may have other feature or properties as illustrated and described in U.S. Patent Application Publication No. 2020/0038048 (Ebersole et al.), previously incorporated above.

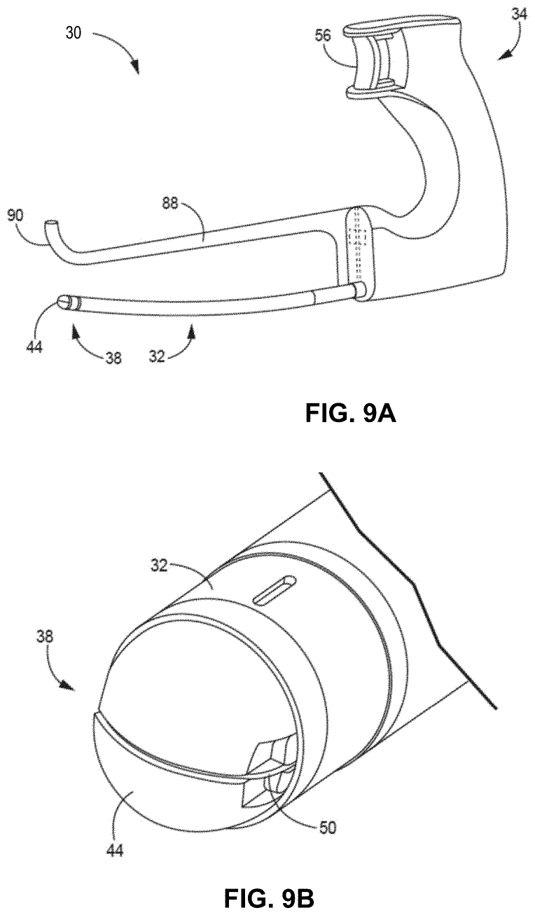

FIGS. 9A-9C are conceptual diagrams illustrating bipolar tunneling tool 30. FIG. 9B illustrates distal end 38 of tunneling shaft 32, which includes optical window 44 and active electrode 50 (shown in the recessed position). FIG. 9C illustrates a view of bipolar tunneling tool 30 similar to that of FIG. 9A but with an outer portion of handle 34 removed to show, among others, the mechanism employed to allow trigger 56 to be actuated such that mechanical and/or electrical energy is translated active electrode 50, e.g., to selectively deploy active electrode 50 as an electrosurgery implement for dissection of tissue. As shown in FIGS. 9A-9C, bipolar tunneling tool 30 may include guide member 88 extending from handle 34 adjacent to and coplanar with tunneling shaft 32. Tunneling shaft 32 may be curved towards guide member 88. Handle 34 has a shape configured to receive fingers of a hand of a surgeon or other operator but may have any other suitable configuration for gripping. When gripped by the surgeon or other operator, a finger such as the index finger may be located in a manner that allows the finger to easily depress and release trigger 56.

During a procedure to gain sub-sternal access and create a sub-sternal tunnel in a patient, guide member 88 may help a surgeon or other operator in advancing tunneling shaft 32, once distal end 38 is inserted into patient 12. In some examples, curved distal portion 90 of guide member 88 may be configured to ‘ride’ on the skin over the sternum 13 without binding on the skin during such a procedure. In this manner, guide member 88 may limit the depth below the sternum 13 that tunneling shaft 32 may be advanced during the tunneling procedure. Further, the curvature of tunneling shaft 32 toward guide member 88 can cause distal end 38 to ‘ride’ adjacent an inside surface of sternum 13 during the superior advancement thereof as an additional aid to the operator. In some example, the distance 93 between guide member 88 and tunneling shaft 32 may be adjusted as desired by a surgeon or other operator, e.g., based on the physical characteristics of a patient. Examples of guide members 88 employed in a tunneling tool may include those described in U.S. patent application Ser. No. 15/204,579, by Malewicz et al., the entire content of which is incorporated herein by reference.

As shown in FIG. 9C, handle 34 includes an elastic member like a cable, chain or belt 95 that is coupled to trigger 56. When trigger is pulled, the elastic member drives the slider 57 which in turn activates the hammer mechanism deploying momentarily the active electrode 50 to cut through adhesions. The active electrode 50 retracts automatically as in the mechanism described above.

In some examples, an endoscope or other tool be may be inserted into proximal opening 48 of handle 34 and advanced, e.g., through inner lumen 46 to or near distal end 38 of tunneling shaft 32. Tunneling shaft 32 may include inner lumen 46 that is open and may accept a rigid or flexible scope through proximal opening 48 in handle 34. Distal end 38 of tunneling shaft 32 may be made of clear or otherwise transparent material so as to enable direct visualization of the tissue through which tunneling shaft 32 is advancing. Active electrode 50 may be recessed (e.g., at least partially disposed) in a channel at distal end 38 when in a recessed position. Tunneling shaft 32 may also include additional lumens, which may be used, e.g., for insufflation, irrigation, adding contrast, flushing lens, removing air pockets, and the like. Bipolar tunneling tool 30 may have other feature or properties as illustrated and described in U.S. Patent Application Publication No. 2020/0038048 (Ebersole et al.), previously incorporated above.

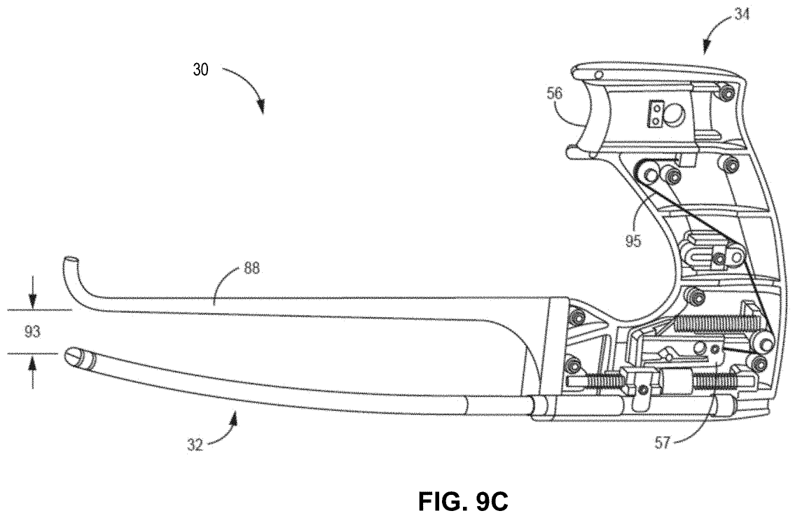

FIG. 10 is a conceptual diagram of distal end 38 of bipolar tunneling tool 30. As described above and shown in FIG. 10, distal end 38 of tunneling shaft 32 includes a return electrode 52. Return electrode 52 is configured to provide a robust 360° return path during electrical activation of active electrode 50 to complete an electrical circuit. For example, distal end 38 may define return electrode 52 in the shape of a cuff.

Distal end 38 may include optical window 44. Optical window 44 may be generally bulbous and define a blunt tip configured to facilitate dissection by dissecting tissue is an atraumatic manner. A channel 108 is defined along optical window 44 and is configured to house a first portion 102 (which may be thin and wire-like) of active electrode 50 such that at least first portion 102 of active electrode 50 extends across optical window 44 substantially along a center-line of optical window 44. Active electrode 50 may be secured at both ends by an insulative housing 106 that electrically insulates active electrode 50 from return electrode 52. Insulative housing 106 may be configured to sit within a recess 100 defined in a side of optical window 44 such that a second portion 104 (which may be larger than first portion 102) of active electrode 50 is exposed. Second portion 104 of active electrode 50 on the side of optical window 44 within recess 100 may facilitate greater tissue dissection compared to first portion 102 of active electrode 50 that extends across distal end 38, which promotes blunt dissection as bipolar tunneling device 30 tunnels through tissue.

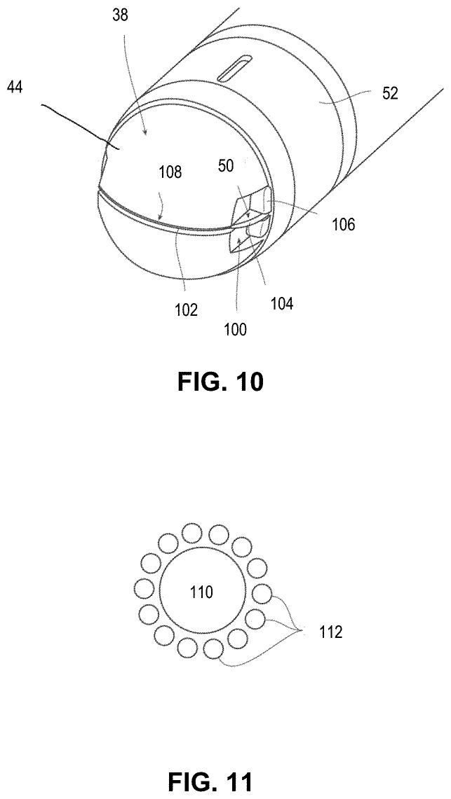

FIG. 11 is a conceptual diagram showing one possible arrangement of a lighting element 112 disposed proximate a camera 110 of bipolar tunneling tool 30. In some examples, lighting element 112 may be disposed proximate optical window 44. Lighting element 112 may be configured to emit light through optical window 44. Camera 110 may be disposed proximate optical window 44; lighting element 112 may be disposed proximate camera 110. In the example of FIG. 11, lighting element 112 is a fiber optic array encircling camera 110. Camera 110 may be mounted to tunneling shaft 32 in any suitable way Camera 110 may be centrically disposed between lighting element 112 or may be off-center depending upon a particular purpose. Other configurations of bipolar tunneling tool 30 are envisioned.

Camera 110 may he selectively moveable relative to tunneling shaft 32 and/or lighting element 112 via one or more actuators (not shown) disposed on handle 34. Lighting element 112 may be configured to selectively emit light therefrom in varying intensities via one or more actuators (not shown) or may be configured to automatically adjust the light setting based on an algorithm or generator setting. Lighting element 112 may be configured to turn on with camera 110 or independently. Lighting element 112 may be configured to connect to a generator that supplies energy thereto. Camera 110 may be configured to connect to a display (not shown).

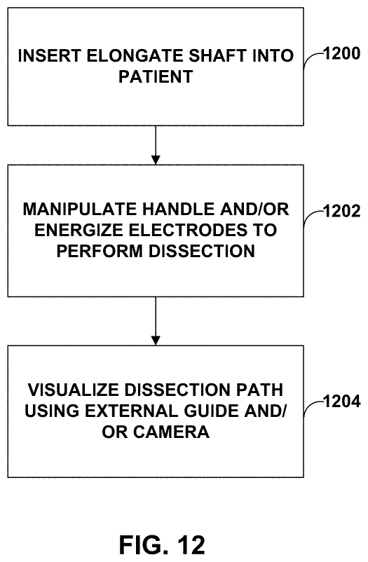

FIG. 12 is a flow diagram of an example technique for operating bipolar tunneling tool 30. In operation according to one example, a user inserts tunneling shaft 32 of bipolar tunneling tool 30 into a patient proximate the sternum (or some other part of the patient's body) and manually orients distal end 38 beneath the skin while maintaining guide member 88 outside the skin above the sternum (1200).

The user may manipulate (e.g., rotate, push, pull, etc.) handle 34 and activate active electrode 50 (e.g., by actuating trigger 56) as needed to perform dissection (1202). For example, the user may push bipolar tunneling tool 30 distally to dissect tissue while activating active electrode 50 to break stubborn tissue areas and scar tissue. The user may also activate active electrode 50 to cauterize tissue, thereby stemming bleeding that would otherwise obstruct visualization via optical window 44. The tissue is dissected proximate active electrode 50 and current is returned through return electrode 52 completing the electrical circuit.

The user may visualize the dissection path during operation of bipolar tunneling tool 30 using guide member 88 and/or camera 110 (1204). The user may externally visualize the internal path of dissection of bipolar tunneling tool 30 by looking at guide member 88, which is moving along or above an outer surface of the sternum of the patient in an extracorporeal fashion and in a substantially parallel fashion to tunneling shaft 32. Additionally or alternatively, the user may refer to a display connected to camera 110. Camera 110 and/or lighting element 112 may be selectively adjusted as needed (e.g., via one or more actuators disposed on handle 34) to ensure adequate visualization during dissection of tissue.

The following examples are illustrative of the techniques described herein.

Example 1: A bipolar tunneling tool includes a handle; a tunneling shaft extending from the handle; a return electrode disposed proximate a distal end of the tunneling shaft; an optical window disposed proximate the distal end of the tunneling shaft, the optical window defining a channel; an active electrode at least partially disposed within the channel, the active electrode configured to dissect a tissue and cauterize the tissue; and an insulative housing configured to electrically insulate the active electrode from the return electrode.

Example 2: The bipolar tunneling tool of example 1, wherein the channel extends substantially along a center-line of the optical window.

Example 3: The bipolar tunneling tool of any of examples 1 and 2, wherein the end distal end includes a lighting element and a camera disposed proximate the optical window.

Example 4: The bipolar tunneling tool of any of examples 1 through 3, further including an activation element configured to activate the active electrode in response to actuation of the activation element.

Example 5: The bipolar tunneling tool of example 4, wherein the activation element is positioned on the handle for actuation by one or more of a user's fingers when the user grips the handle.

Example 6: The bipolar tunneling tool of example 5, wherein actuation of the activation element causes the active electrode to extend distally from the channel.

Example 7: The bipolar tunneling tool of any of examples 1 through 6, wherein the tunneling shaft extends in a curved orientation from a proximal end of the tunneling shaft to the distal end of the tunneling shaft.

Example 8: The bipolar tunneling tool of any of examples 1 through 7, wherein the optical window includes a blunt tip to dissect the tissue in an atraumatic manner,

Example 9: The bipolar tunneling tool of any of examples 1 through 8, wherein the active electrode includes a first portion extending across the optical window and a second portion disposed within a recess defined in a side of the optical window.

Example 10: The bipolar tunneling tool of any of examples 1 through 9, further including a guide member extending from the handle that is adjacent to and coplanar with the tunneling shaft.

Example 11: The bipolar tunneling tool of example 10, wherein the guide member includes a distal end that is curved away from a sternum during use of the bipolar tunneling tool.

Example 12: The bipolar tunneling tool of any of examples 1 through 11, wherein the lighting element is a fiber optic array encircling the camera.

Example 13: A bipolar tunneling tool includes a handle including an activation element; a tunneling shaft extending from the handle; a return electrode disposed proximate a distal end of the tunneling shaft; an active electrode disposed at the distal end of the tunneling shaft, wherein the activation element is configured to activate the active electrode in response to actuation of the activation element, and wherein the active electrode is configured to dissect a tissue and cauterize the tissue; an insulative housing configured to electrically insulate the active electrode from the return electrode; and a guide member extending from the handle in a substantially parallel fashion to the tunneling shaft, the guide member configured to move above an outer surface of a sternum in an extracorporeal fashion during use of the bipolar tunneling tool.

Example 14: The bipolar tunneling tool of example 13, further including an optical window at the distal end of the tunneling shaft, the optical window defining a channel, wherein the active electrode is at least partially disposed within the channel.

Example 15: The bipolar tunneling tool of example 14, wherein the bipolar tunneling tool includes a lighting element and a camera disposed proximate the optical window.

Example 16: The bipolar tunneling tool of any of examples 14 and 15, wherein the active electrode includes a first portion extending across the optical window and a second portion disposed within a recess defined in a side of the optical window.

Example 17: The bipolar tunneling tool of any of examples 13 through 16, wherein actuation of the activation element causes the active electrode to extend distally from the channel.

Example 18: The bipolar tunneling tool of any of examples 13 through 17, wherein the optical window includes a blunt tip to dissect the tissue in an atraumatic manner.

Example 19: A method includes inserting a tunneling shaft of a bipolar tunneling tool into a substernal space of a patient, wherein the bipolar tunneling tool includes: a return electrode disposed proximate a distal end of the tunneling shaft; an optical window disposed proximate the return electrode, the optical window defining a channel; an active electrode at least partially disposed within the channel, the active electrode configured to dissect a tissue and cauterize the tissue; an insulative housing configured to electrically insulate the active electrode from the return electrode; and a camera disposed proximate the optical window and configured to connect to a display; actuating an activation element configured to activate the active electrode in response to actuation of the activation element; and visualizing a dissection path of the bipolar tunneling tool using the camera.

Example 20: The method of example 19, wherein the bipolar tunneling tool further includes a guide member extending from a handle of the bipolar tunneling tool, wherein the guide member is configured to move above an outer surface of a sternum in an extracorporeal fashion during use of the bipolar tunneling, and wherein visualizing a dissection path of the bipolar tunneling tool further includes using the guide member to externally visualize the dissection path.

Various examples have been described. These and other examples are within the scope of the following claims.

What is claimed is:

1. A bipolar tunneling tool comprising:
a handle;
a tunneling shaft extending from the handle;
a return electrode disposed proximate a distal end of the tunneling shaft;
an optical window disposed proximate the distal end of the tunneling shaft, the optical window defining a channel;
an active electrode at least partially disposed within the channel, the active electrode configured to dissect a tissue and cauterize the tissue; and
an insulative housing configured to electrically insulate the active electrode from the return electrode.

2. The bipolar tunneling tool of claim 1, wherein the channel extends substantially along a center-line of the optical window.

3. The bipolar tunneling tool of claim 1, wherein the end distal end comprises a lighting element and a camera disposed proximate the optical window.

4. The bipolar tunneling tool of claim 1, further comprising an activation element configured to activate the active electrode in response to actuation of the activation element.

5. The bipolar tunneling tool of claim 4, wherein the activation element is positioned on the handle for actuation by one or more of a user's fingers when the user grips the handle.

6. The bipolar tunneling tool of claim 5, wherein actuation of the activation element causes the active electrode to extend distally from the channel.

7. The bipolar tunneling tool of claim 1, wherein the tunneling shaft extends in a curved. orientation from a proximal end of the tunneling shaft to the distal end of the tunneling shaft.

8. The bipolar tunneling tool of claim 1, wherein the optical window includes a blunt tip to dissect the tissue in an atraumatic manner.

9. The bipolar tunneling tool of claim 1, wherein the active electrode comprises a first portion extending across the optical window and a second portion disposed within a recess defined in a side of the optical window.

10. The bipolar tunneling tool of claim 1, further comprising a guide member extending from the handle that is adjacent to and coplanar with the tunneling shaft.

11. The bipolar tunneling tool of claim 10, wherein the guide member comprises a distal end that is curved away from a sternum during use of the bipolar tunneling tool.

12. The bipolar tunneling tool of claim 1, wherein the lighting element is a fiber optic array encircling the camera.

13. A bipolar tunneling tool comprising:
a handle comprising an activation element;
a tunneling shaft extending from the handle;
a return electrode disposed proximate a distal end of the tunneling shaft
an active electrode disposed at the distal end of the tunneling shaft, wherein the activation element is configured to activate the active electrode in response to actuation of the activation element, and wherein the active electrode is configured to dissect a tissue and cauterize the tissue;
an insulative housing configured to electrically insulate the active electrode from the return electrode; and
a guide member extending from the handle in a substantially parallel fashion to the tunneling shaft, the guide member configured to move above an outer surface of a sternum in an extracorporeal fashion during use of the bipolar tunneling tool.

14. The bipolar tunneling tool of claim 13, further comprising an optical window at the distal end of the tunneling shaft, the optical window defining a channel, wherein the active electrode is at least partially disposed within the channel.

15. The bipolar tunneling tool of claim 14, wherein the bipolar tunneling tool comprises a lighting element and a camera disposed proximate the optical window.

16. The bipolar tunneling tool of claim 14, wherein the active electrode comprises a first portion extending across the optical window and a second portion disposed within a recess defined in a side of the optical window.

17. The bipolar tunneling tool of claim 13, wherein actuation of the activation element causes the active electrode to extend distally from the channel.

18. The bipolar tunneling tool of claim 13, wherein the optical window comprises a blunt tip to dissect the tissue in an atraumatic manner.

19. A method comprising:
inserting a tunneling shaft of a bipolar tunneling tool into a substernal space of a patient, wherein the bipolar tunneling tool comprises:
a return electrode disposed proximate a distal end of the tunneling shaft;
an optical window disposed proximate the return electrode, the optical window defining a channel;
an active electrode at least partially disposed within the channel, the active electrode configured to dissect a tissue and cauterize the tissue;
an insulative housing configured to electrically insulate the active electrode from the return electrode; and
a camera disposed proximate the optical window and configured to connect to a display;
actuating an activation element configured to activate the active electrode in response to actuation of the activation element; and
visualizing a dissection path of the bipolar tunneling tool using the camera.

20. The method of claim 19, wherein the bipolar tunneling tool further comprises a guide member extending from a handle of the bipolar tunneling tool, wherein the guide member is configured to move above an outer surface of a sternum in an extracorporeal fashion during use of the bipolar tunneling, and wherein visualizing a dissection path of the bipolar tunneling tool further comprises using the guide member to externally visualize the dissection path.
